(19) United States
(12) Patent Application Publication
(10) Pub. No.: US 2025/0303413 A1
(43) Pub. Date: Oct. 2, 2025

(54) Methods, Systems, and Devices for Ear Mite Analysis

(71) Applicant: IDEXX Laboratories, Inc., Westbrook, ME (US)

(72) Inventors: Jeremy Hammond, Westbrook, ME (US); Jason Aguiar, Westbrook, ME (US); JuiMing Lin, Westbrook, ME (US); Richard Andrews, Westbrook, ME (US); David Connolly, Westbrook, ME (US); Rachel Bowman, Westbrook, ME (US); James Russell, Westbrook, ME (US)

(21) Appl. No.: 19/094,359

(22) Filed: Mar. 28, 2025

Related U.S. Application Data
(60) Provisional application No. 63/572,326, filed on Mar. 31, 2024.

(51) Int. Cl. B01L 3/00

(52) U.S. Cl. CPC B01L 3/502715; B01L 2200/04; B01L 2200/16; B01L 2300/0654

(57) ABSTRACT

A method for evaluating a biological sample is disclosed. The method includes: (i) receiving the biological sample in a container comprising a first volume, wherein the container comprises a diluent mixture comprising a diluent and a surfactant; (ii) generating a biological testing sample in the container, wherein the biological testing sample comprises the biological sample and the diluent mixture, and wherein the biological testing sample comprises a second volume; (iii) depositing the biological testing sample from the container into a reservoir of a cartridge, wherein the reservoir is configured to receive the biological testing sample, and wherein the reservoir comprises a third volume; and (iv) analyzing the biological testing sample, wherein analyzing the biological testing sample comprises capturing one or more images of the biological testing sample from an imaging sensor of an imaging device.

### RELATED APPLICATION

This application claims priority to U.S. Provisional Application No. 63/572,326 filed on Mar. 31, 2024, which is incorporated herein by reference in its entirety.

### FIELD OF THE DISCLOSURE

The present disclosure involves devices and systems for evaluating a biological sample comprising ear mites, and methods for using and manufacturing the devices and systems thereof. Namely, devices, systems, and methods of the present disclosure involve a preparing a biological testing sample in a container and transferring the prepared biological testing sample to a cartridge for analysis, such as digital imaging.

### BACKGROUND

Testing and imaging of biological samples can be conducted utilizing a variety of different systems and methods. For example, a cartridge, compatible with an imaging device, can provide one or more areas for containing and/or imaging a biological sample (e.g., in a reservoir).

### SUMMARY

In another example, a method of evaluating a biological sample comprising ear mites is disclosed. The method includes: (i) receiving the biological sample in a container comprising a first volume, wherein the container comprises a diluent mixture comprising a diluent and a surfactant; (ii) generating a biological testing sample in the container, wherein the biological testing sample comprises the biological sample and the diluent mixture, and wherein the biological testing sample comprises a second volume; (iii) depositing the biological testing sample from the container into a reservoir of a cartridge, wherein the reservoir is configured to receive the biological testing sample, and wherein the reservoir comprises a third volume; and (iv) analyzing the biological testing sample, wherein analyzing the biological testing sample comprises capturing one or more images of the biological testing sample from an imaging sensor of an imaging device.

In another example, an example non-transitory computer-readable medium that, upon execution by a computing device, cause the computing device to perform a set of operations for evaluating a biological sample comprising ear mites is disclosed. The set of operations includes: (i) receiving the biological sample in a container comprising a first volume, wherein the container comprises a diluent mixture comprising a diluent and a surfactant; (ii) generating a biological testing sample in the container, wherein the biological testing sample comprises the biological sample and the diluent mixture, and wherein the biological testing sample comprises a second volume; (iii) depositing the biological testing sample from the container into a reservoir of a cartridge, wherein the reservoir is configured to receive the biological testing sample, and wherein the reservoir comprises a third volume; and (iv) analyzing the biological testing sample, wherein analyzing the biological testing sample comprises capturing one or more images of the biological testing sample from an imaging sensor of an imaging device.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional features will be better understood through the following illustrative and non-limiting detailed description of example embodiments, with reference to the appended drawings.

FIG. 1 illustrates a simplified block diagram of an example computing device, according to an example embodiment.

FIG. 2 illustrates an example container, according to an example embodiment.

FIG. 3 illustrates an example cartridge, before assembly of the cartridge, according to an example embodiment.

FIG. 4A illustrates an example cartridge, before assembly of the cartridge, according to an example embodiment.

FIG. 4B illustrates the example cartridge of FIG. 4A from an opposing view, according to an example embodiment.

FIG. 4C illustrates the example cartridge of FIGS. 4A-4B, after assembly of the cartridge, according to an example embodiment.

FIG. 5 illustrates an example computing system, according to an example embodiment.

FIG. 6 illustrates experimental results, according to an example embodiment.

FIG. 7 illustrates experimental results, according to an example embodiment.

FIG. 8 illustrates a method, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Within examples, the present disclosure is directed to devices and systems for imaging and/or otherwise analyzing a biological sample comprising ear mites.

Testing and/or imaging, as referred to herein, may include, for example, capturing one or more images related to a sample. For example, testing can involve capturing images of a biological sample from an imaging sensor and determining one or more parameters of the biological sample and/or components thereof. One or more machine learning models can then be implemented to analyze the captured images and perform one or more computational actions, including identifying a characteristic of the biological sample. In some examples, computer vision techniques can also be employed to identify and process characteristics of a biological sample in an image.

Generally, preparing a biological sample for such testing involves mixing the biological sample into a liquid diluent and introducing one or more reagents to the mixture of the biological sample and the liquid diluent prior to imaging, testing, and/or other analytical methods. The combination of the biological sample, the liquid diluent, and the reagent(s) can be used to form a biological testing sample, which can then be deposited onto a testing surface (e.g., a slide) or cartridge for testing, such as imaging.

To date, such devices and methods for preparing a biological testing sample require significant manual user handling. Historically, preparation of a biological sample for testing involves a user (e.g., a clinician) manually measuring and handling the liquid diluent to be mixed with the biological sample. Similarly, the preparation may involve the user manually handling and measuring a liquid reagent to agitate and mix with the liquid diluent and biological sample. This process can be time intensive, result in user error in measurement and handling, and produce waste from these potential user errors.

Furthermore, in the context of ear cytology, several additional challenges are presented that lead to user errors and unproductive protocols. For example, ear cytology traditionally occurs by swabbing the ear with a cotton swab and then rolling the swab onto a glass slide, staining it, and then evaluating under a microscope. Common elements to review include red blood cells, white blood cells, bacteria, yeast, and mites or mite ova.

When developing the protocols and components (e.g., reagents) to support ear cytology, it is often difficult to develop them so that these protocols and components do not destroy (lyse) any of the cells or other components of interest in the biological testing sample, including ear mites. Generally, this protocol requires managing the osmolality and pH of the biological testing sample, as well as the type and concentration of other chemicals in the protocol (e.g., reagents). In addition, when incorporating stains into the reagents (e.g., bright field or fluorescent), it is important to match them with the rest of the reagents so that stain uptake is optimized to the application. Further, when evaluating for ear mites in particular, it is important to transfer a complete representation of all of various components of the sample from the swab to a testing platform (e.g. a cartridge) where the microscopy imaging will take place, including by transferring cells and ear mites from a swab to a glass slide for microscopy evaluation and/or transferring the sample components from the swab to a diluent and then to a cartridge for microscopy analysis.

In the context of transferring a biological sample comprising ear mites into a diluent prior to imaging, additional challenges have been identified, including that ear mites often float in diluents and therefore can pose difficulty in transferring them from the diluent to a testing platform (e.g. a cartridge). Furthermore, difficulty associated with floating ear mites is not resolved by pouring techniques, which can cause the floating mites to migrate to the highest point in the fluid, away from the pouring area. Similarly, dropper and other dispense techniques of a disposable will generally be placed at the lowest point of the fluid, and therefore the floating mites will be farthest from the dispense area and will most likely be missed in the transfer.

In addition to floating, ear mites tend to stick to surfaces, which can result in an incomplete transfer of all of the mites in a diluent from a diluent/reagent container to a cartridge or other testing platform. This challenge has been demonstrated with both living and dead mites, so it is not an active function that the mite performs to hold on to a surface, and instead is an inherent physical condition on the exterior surface of the mite and container walls that supports the adherence.

The example systems, devices, and methods disclosed herein address these challenges. An example method of the present disclosure includes a container configured to receive a biological sample, mix the biological sample with a liquid diluent and a surfactant, introducing a reagent to the biological sample and diluent mixture, generating a biological testing sample in the container that contains the biological sample and the diluent mixture, and then dispensing the biological testing sample containing the biological sample, diluent, and reagent onto a testing surface. To do so, the container may include a dispensing nozzle for dispensing the biological testing sample (e.g., the mixture of the diluent, the biological sample, and the reagent) onto the testing surface, which includes a cartridge with a reservoir for containing a biological sample. Further, in some examples, the volume of the container may be configured to match or approximately match (e.g., within a 98% or 99% tolerance) the volume of the biological testing sample (including the combined volumes of the biological sample, diluent, and reagent). Furthermore, one or more additional materials may be introduced into the biological testing sample to further alleviate one or more issues that arise from ear mite analysis, including coating of one or more walls of the container and/or cartridge reservoir with one or more materials to reduce ear mites from adhering to surfaces of the container and/or cartridge reservoir and therefore improve transfer to the imaging cartridge. Additionally or alternatively, one or more surfactants may be added to the diluent and/or other components of the biological testing sample to inhibit ear mites from adhering to surfaces of the container and/or cartridge reservoir and thus similarly improve transfer to the imaging cartridge.

In other example embodiments, different volumes of a reservoir in the cartridge can have advantages depending on the type of biological sample being tested, the concentration of cells within a biological sample, and/or the type of testing performed. For instance, the volume of the reservoir may be configured to match or approximately match (e.g., within a 98% or 99% tolerance) the volume of the dispensed biological testing sample. As such, imaging of a biological testing sample may be more uniform which can improve consistency, accuracy, and repeatability of tests. The example containers, cartridges, and methods described herein also improve precision and consistency of one or more parameters of the testing protocols described herein, and may lead to improved imaging techniques and diagnostic results (e.g., ear mite detection results), alike.

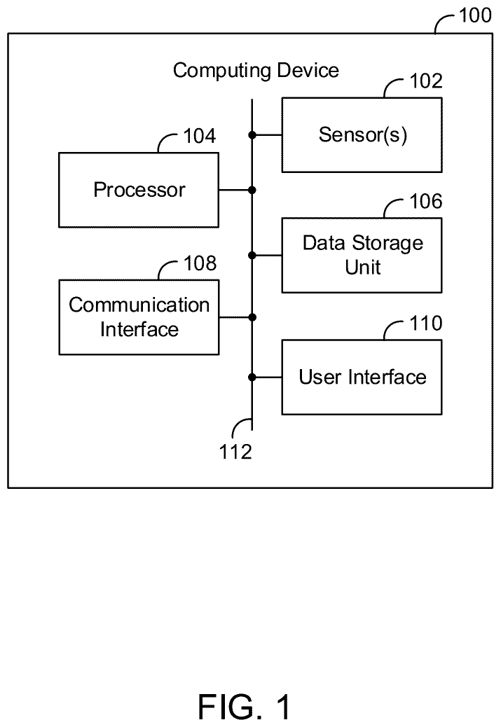

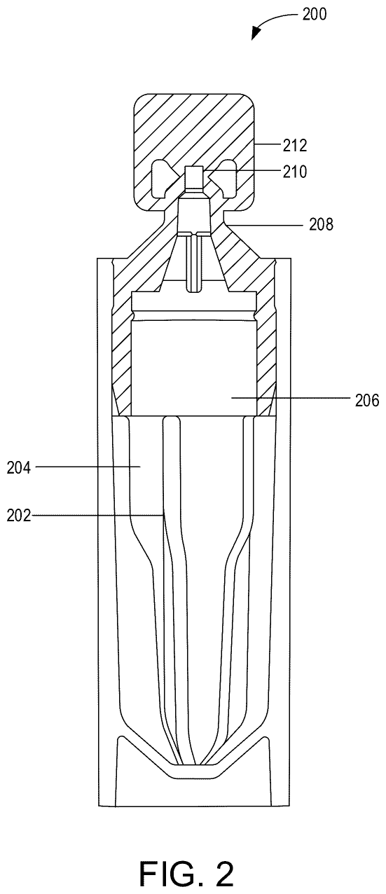

Referring now to the figures, FIG. 1 is a simplified block diagram of an example computing device 100 of a system (e.g., that can be utilized with devices and methods illustrated in FIGS. 2-7, described in further detail below). Computing device 100 can perform various acts and/or functions, such as those described in this disclosure. Computing device 100 can include various components, such as processor 102, data storage unit 104, communication interface 106, and/or user interface 108. These components can be connected to each other (or to another device, system, or other entity) via connection mechanism 110.

Processor 102 can include a general-purpose processor (e.g., a microprocessor and/or a central processing unit (CPU)) and/or a special-purpose processor (e.g., a digital signal processor (DSP) and/or a graphics processing unit (GPU)).

Data storage unit 104 can include one or more volatile, non-volatile, removable, and/or non-removable storage components, such as magnetic, optical, or flash storage, and/or can be integrated in whole or in part with processor 102. Further, data storage unit 104 can take the form of a non-transitory computer-readable storage medium, having stored thereon program instructions (e.g., compiled or non-compiled program logic and/or machine code) that, when executed by processor 102, cause computing device 100 to perform one or more acts and/or functions, such as those described in this disclosure. As such, computing device 100 can be configured to perform one or more acts and/or functions, such as those described in this disclosure. Such program instructions can define and/or be part of a discrete software application. In some instances, computing device 100 can execute program instructions in response to receiving an input, such as from communication interface 106 and/or user interface 108. Data storage unit 104 can also store other types of data, such as those types described in this disclosure.

Communication interface 106 can allow computing device 100 to connect to and/or communicate with another other entity according to one or more protocols. In one example, communication interface 106 can be a wired interface, such as an Ethernet interface or a high-definition serial-digital-interface (HD-SDI). In another example, communication interface 106 can be a wireless interface, such as a cellular or WI FI interface. In this disclosure, a connection can be a direct connection or an indirect connection, the latter being a connection that passes through and/or traverses one or more entities, such as a router, switcher, or other network device. Likewise, in this disclosure, a transmission can be a direct transmission or an indirect transmission.

User interface 108 can facilitate interaction between computing device 100 and a user of computing device 100, if applicable. As such, user interface 108 can include input components such as a keyboard, a keypad, a mouse, a touch sensitive panel, a microphone, a camera, and/or a movement sensor, all of which can be used to obtain data indicative of an environment of computing device 100, and/or output components such as a display device (which, for example, can be combined with a touch sensitive panel), a sound speaker, and/or a haptic feedback system. More generally, user interface 108 can include hardware and/or software components that facilitate interaction between computing device 100 and the user of the computing device 100.

Computing device 100 can take various forms, such as a workstation terminal, a desktop computer, a laptop, a tablet, a mobile phone, or a controller.

Now referring to FIG. 2, a container 200 for preparing and dispensing a biological testing sample is illustrated, according to an example embodiment. The container 200 includes extraction ribs 202 that can remove solid biological samples (e.g., ear wax) from one or more tools (e.g., one or more swabs) that are inserted into the diluent chamber 204 of container 200. To further facilitate depositing the biological sample in the diluent chamber 204 of container 200, one or more substances may be deposited into the diluent chamber 204, including one or more liquid diluents (e.g., water), as well as one or more surfactants (e.g., an anionic surfactant), including for example, dioctyl sulfosuccinate (DOSS) and sodium dodecyl sulfate (SDS), among other possibilities. In some examples, the diluent mixture comprises about 0.005% to about 0.02% dioctyl sulfosuccinate, for example about 0.005%, 0.01%, 0.015% and 0.02% dioctyl sulfosuccinate. In another embodiment, the diluent mixture includes 0.05% to about 1.0% SDS, for example about 0.05%, 0.1%, 0.2%, 0.3%, 0.4%, 0.5%, 0.75% or 1.0% SDS.

As also shown in FIG. 2, the diluent chamber 204 is in fluid communication with the reagent chamber 206, allowing the reagent to mix with the liquid diluent, surfactant, and the biological sample. The container 200 can be handled, for example, inverted and/or shaken, to agitate the reagent and mix it with the liquid diluent, surfactant, and the biological sample to form the biological testing sample. In examples where the reagent is a solid lyophilized reagent, agitation of the reagent with the liquid diluent can liquefy the reagent. This allows the reagent, liquid diluent, and biological sample to mix and prepare the biological testing sample for dispensing and testing. During such handling, spillage or leakage of the fluids (e.g., the liquid diluent, the biological sample, and the reagent) is prevented by way of the liquid seal created by the interface of the illustrated components of FIG. 2.

Once the biological testing sample is prepared, the biological testing sample can be dispensed onto a testing surface (e.g., into a reservoir of a cartridge) by way of the dispensing nozzle 208. The dispensing nozzle 208 is in fluid communication with the reagent chamber 206 and the diluent chamber 204, which together may form a volume that is approximately the same volume as the prepared biological testing sample (e.g., approximately 0.5 milliliters). In examples, the dispensing nozzle includes an opening 210, the opening 210 being sealed until the biological testing sample is prepared. In examples, the opening 210 is sealed by way of a break-away tab 212.

In examples, when the break-away tab 212 is removed and the opening 210 of the dispensing nozzle 208 is exposed, the biological testing sample can be dispensed from the container 200 onto a testing surface, such as a reservoir of a cartridge. In examples, the opening of the dispensing nozzle 208 is configured to dispense the biological testing sample at a particular flow rate. More particularly, the opening 210 has a particular cross-sectional area to achieve a desired flow rate of the biological testing sample, which can help control the placement of the biological testing sample onto the testing surface and prevent splatter.

Additionally, as noted above, the container 200 can be made of a compliant material (e.g., polyolefin elastomer (POE), ethylene-vinyl acetate (EVA) copolymer, LLDPE, and/or LDPE) so that the user can pinch or squeeze the container 200 to dispense the biological testing sample onto the testing surface. In example embodiments, the compliant material is configured to dispense the biological testing sample at a particular flow rate. This helps further control placement of the biological testing sample onto the testing surface and prevent splatter.

In some example embodiments, the biological testing sample can be used for a variety of tests. For instance, these tests may include imaging of one or more of the following: (i) blood; (ii) urine; (iii) saliva; (iv) fecal matter; (v) secretion; (vi) excretion; (vii) fine needle aspirate; (viii) lavage fluids; (ix) body cavity fluids; (x) semen; (xi) ear wax; (xii) skin cells; (xiii) biopsied samples, (xiv) exotics; (xv) cultured cells; (xvi) bacteria; (xvii) worms; and (xviii) parasites, among other possibilities.

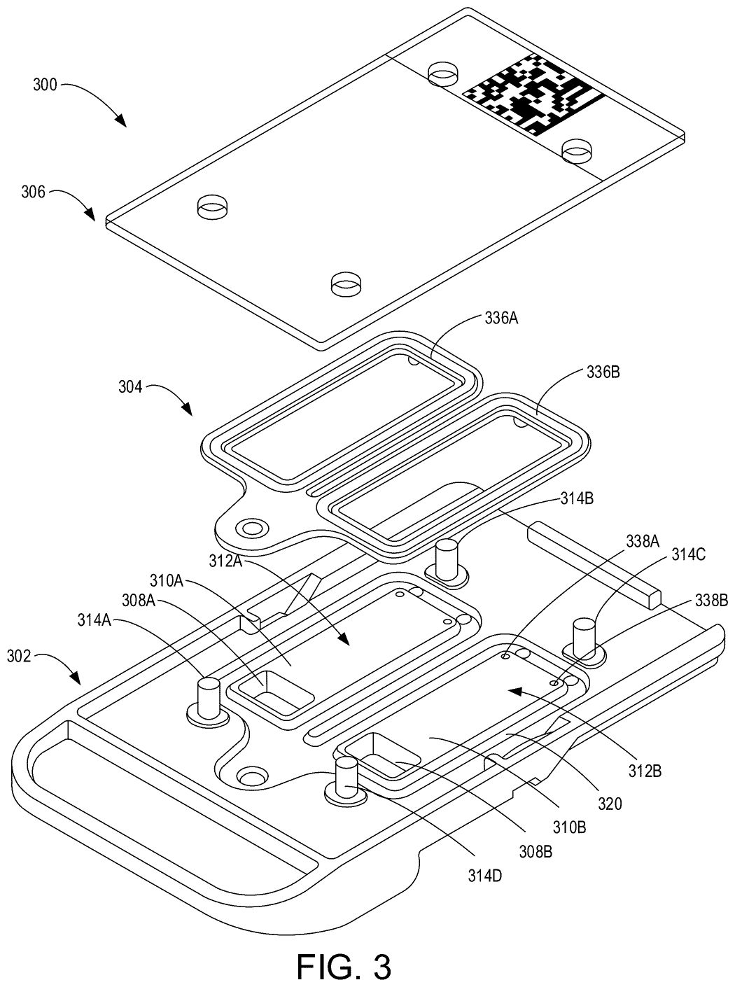

Referring now to FIG. 3, which illustrates an example cartridge in an exploded view prior to assembly. An example cartridge includes a housing, a gasket, and a sheet. Once the example cartridge is assembled, components of the housing, the gasket, and the sheet form boundaries of a reservoir. The reservoir is configured to contain a biological testing sample during testing.

FIG. 3 illustrates a housing 302, a gasket 304, and a sheet 306, before assembly of the cartridge 300, according to an example embodiment.

In example embodiments the housing 302 includes a first port 308A and a second port 308B. The first port 308A and second port 308B include apertures suitable for receiving a biological testing sample. For instance, in example embodiments, the first port 308A and second port 308B are openings that are large enough for a user to insert the biological testing sample (e.g., by inserting nozzle of container 200) into the first port 308A and/or second port 308B. Many example configurations are possible. Further, in examples, the first port 308A and second port 308B are of a sufficient depth to avoid backflow of the biological testing sample once inserted into the cartridge 300.

In example embodiments, the housing 302 includes a first recess 310A and a second recess 310B. When the cartridge 300 is assembled, the first port 308A is in fluid communication with a first reservoir 312A and the second port 308B is in fluid communication with a second reservoir 312B. In these examples, when the gasket 304 is disposed in a channel 320 of the housing 302 (as shown in FIGS. 3B and 3C), boundaries of a first reservoir 312A are defined by the first recess 310A, the first wall 336A of the gasket 304A, and a surface of sheet 306 shown. In examples, the height of the first reservoir 312A may be different than the height of the second reservoir 312B. In examples, having two reservoirs of two different heights allows for performing multiple tests at once on a biological testing sample. In a further aspect, one or more of the illustrated reservoirs may comprises one or more vents, including first vent 338A and second vent 338B. In example embodiments, first vent 338A and second vent 338B may promote fluid communication within the reservoir, as first vent 338A and/or second vent 338B promote a fluid (e.g., a biological testing sample) dispersing throughout the reservoir after being inserted and dispersed into second port 308B. Further, in examples, the first vent 338A and second vent 338B may help avoid backflow of the biological testing sample once inserted into the cartridge 300 via second port 308B.

The two-port cartridge 300 facilitates testing of multiple biological samples at once. For instance, in some examples, a user can test ear wax samples from a left ear and a right ear simultaneously. Additionally, a user can deposit a biological sample collected from the same source in both the first reservoir 312A and the second reservoir 312B which allows the user to conduct comparative analyses on biological samples contained in each of the two reservoirs. In examples, this arrangement can provide an improvement over current technologies by, for example, helping reduce testing time and/or the consistency of results and analysis over multiple tests. Further, a user can perform a single testing protocol on of two different biological samples at once. Additionally or alternatively, a user can prepare one biological sample with two separate reagents. In these examples, this allows a user to chemically remove some of the elements in the biological sample or preferentially stain different elements to aid in detection. Further, a user can also perform tests with two different reservoir heights at once. In example implementations, performing multiple tests at once, for instance, two biological samples, can help reduce testing time and consistency among tests.

To facilitate the reception and/or disposal of the gasket 304 in the channel 320, the channel 320 the same or similar cross-sectional shape as the gasket 304. In example embodiments, the gasket 304 and channel 320 may both include two portions to surround the first recess 310A and the second recess 310B, respectively. For instance, the first wall 336A of the gasket 304B can be configured to surround the first recess 310A and the second wall 336B of the gasket 304 can be configured to surround the second recess 310B. In these examples, the walls of the gasket 304 define the boundaries of the first reservoir 312A and the second reservoir 312B. As noted above, having two separate reservoirs 312A and 312B allows for performing multiple tests at once. As noted above, performing multiple tests at once can improve accuracy and efficiency and can, in some examples, allow a user to perform a comparative analysis, if desired.

In some examples, the housing 302 comprises one or more fasteners 314A, 314B, 314C, and 314D. The fasteners 314A, 314B, 314C, and 314D are configured to interface and/or couple the housing 302 to a sheet 306 to assemble the cartridge 300. For instance, in some examples, a sheet 306 can include one or more apertures compatible with the one or more fasteners 314A, 314B, 314C, and 314D. In some example configurations, such as the configuration shown in FIG. 3, the housing 302 can include four fasteners 314A, 314B, 314C, and 314D. Many example configurations of fasteners are possible. For instance, in some examples, the housing 302 can include fewer fasteners (e.g., one, two, or three fasteners). In other examples, the housing 302 can include more fasteners (e.g., five, six, or seven fasteners). In some examples, the fasteners 314A, 314B, 314C, and 314D include one or more datum pads. Additionally or alternatively, the fasteners 314A, 314B, 314C, and 314D can include one or more heat stakes. In some alternative example embodiments, the housing 302 and sheet 306 can be coupled to each other via an adhesive (e.g., a UV cured adhesive).

In examples, the housing 302 includes optically transparent materials suitable for imaging. For instance, some example materials suitable for the housing 302 can include, but are not limited to, glass, acrylic, polystyrene, polypropylene, poly(methyl methacrylate) (PMMA), cyclic olefin copolymer (COC), and cyclic olefin polymer (COP). Many example materials are possible. In example embodiments, the optically transparent housing 302 helps reduce autofluorescence from the housing 302 to provide a clear fluorescent background for imaging.

In example embodiments, the cartridge 300 further includes sheet 306. As noted above, in examples, the sheet 306 can include one or more apertures compatible with the one or more fasteners. Additionally, the sheet can include a label. In some examples, the label includes a machine-readable identifier (e.g., a data matrix code, a barcode, a QR code, etc.). Further, in example embodiments, the label may be a laser-etched label, a printed label, and/or an adhesive label (e.g., a sticker), among other possibilities. In a further aspect, in examples, the QR code may be used to identify one or more medical records associated with sample, the patient, the imaging machine, and/or the testing facility, among other possibilities. For example, the QR code may be used as a verification protocol between the imaging device and the cartridge (e.g., an electronic handshake protocol) to ensure the cartridge is the proper cartridge for the imaging device. Other examples are possible.

In examples, the sheet 306 includes optically transparent materials suitable for imaging. For instance, some example materials suitable for the sheet 306 can include, but are not limited to, glass, acrylic, polystyrene, polypropylene, PMMA, COC, and/or COP. Many example materials are possible. In some example embodiments, the material of the sheet 306 may be the same, or similar, to the material of the housing 302. In other examples, the material of the sheet 306 and the material of the housing 302 can be different from one another.

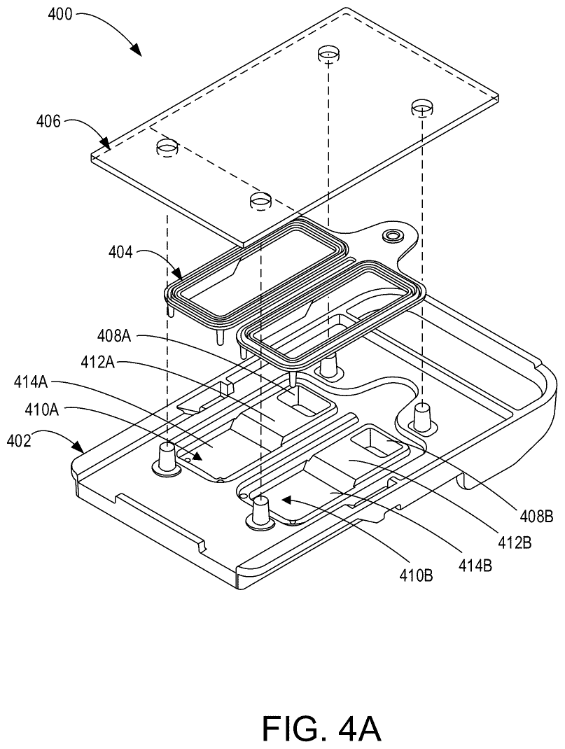

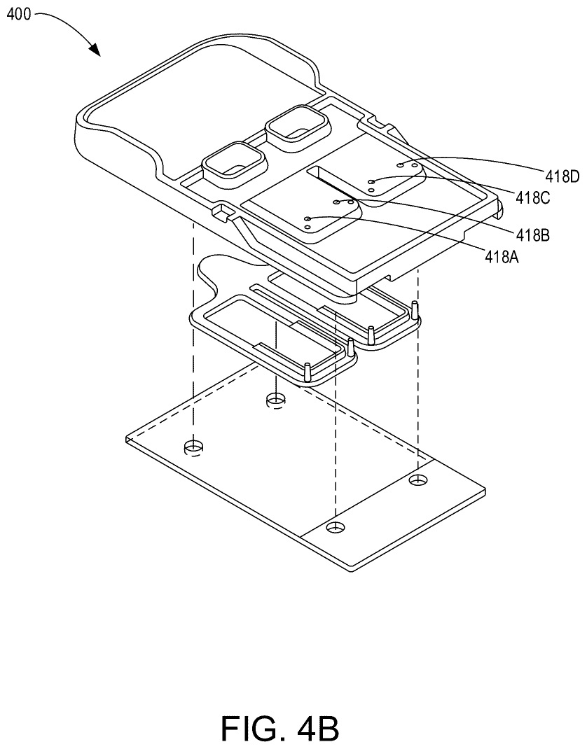

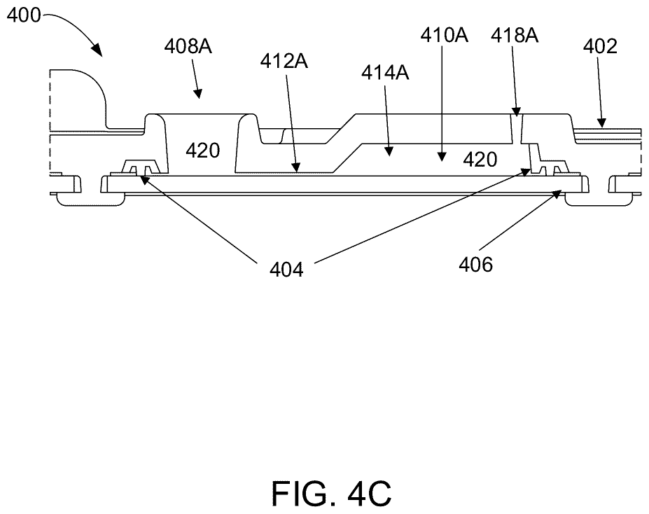

FIGS. 4A-4C illustrate an example cartridge 400 that comprises a housing 402, a gasket 404, and a sheet 406, before assembly of the cartridge 400, according to an example embodiment.

In example embodiments, like cartridge 300, for cartridge 400 the housing 402 includes a first port 408A and a second port 408B. The first port 408A and second port 408B include apertures suitable for receiving a biological testing sample. For instance, in example embodiments, the first port 408A and second port 408B are openings that are large enough for a user to insert the biological testing sample (e.g., by inserting nozzle of container 200) into the first port 408A and/or second port 408B. Many example configurations are possible. Further, in examples, the first port 408A and second port 408B are of a sufficient depth to avoid backflow of the biological testing sample once inserted into the cartridge 400.

In example embodiments, the housing 402 includes a first reservoir 410A that includes a first recess 412A and a second recess 414A and a second reservoir 408B that includes a third recess 412B and a fourth recess 414B. When the cartridge 400 is assembled, the first port 408A is in fluid communication with a first reservoir 410A and the second port 408B is in fluid communication with a second reservoir 410B. In these examples, when the gasket 404 is disposed in a channel 416 of the housing 402, the boundaries of first reservoir 410A are defined by the first recess 412A, the second recess 414A, a portion of gasket 404, and a surface of sheet 406. In these examples, when the gasket 404 is disposed in a channel 416 of the housing 402, the boundaries of second reservoir 410B are defined by the third recess 412B, the fourth recess 414B, another portion of gasket 404, and a surface of sheet 406.

As shown in FIG. 4A, the height of the first reservoir 410A may be different at the first recess 412A and the second recess 414A, and the height of the second reservoir 410B may be different at the third recess 412B and a fourth recess 414B. Additionally or alternatively, any one or more of the first recess 412A, second recess 414A, third recess 412B, and fourth recess 414B may be of the same or different heights, compared to one another. In examples, having two different heights in a single reservoir and/or in different reservoirs allows for performing imaging and/or other analysis at multiple depths in the same reservoir and/or multiple tests at once on a biological testing sample in different reservoirs.

The two-port cartridge 400 also facilitates testing of multiple biological testing samples at once. For instance, in some examples, a user can test ear wax samples (e.g., for ear mites) from a left ear and a right ear simultaneously. Additionally, a user can deposit a biological testing sample collected from the same source in both the first reservoir 410A and the second reservoir 410B which allows the user to conduct comparative analyses on biological testing samples contained in each of the two reservoirs. Furthermore, as shown in FIG. 4A, because each of first reservoir 410A and second reservoir 410B contain two different recess heights, a user can perform testing protocols (e.g., imaging) at multiple depths within the same reservoir on the same the sample. For example, a user may perform a first analysis of a biological testing sample (e.g., capture a first set of images) in reservoir 410A at the shallow portion (corresponding to first recess 412A) and a second analysis of the same biological testing sample (e.g., capture a second set of images) in reservoir 410A at the deeper portion (corresponding to second recess 414A). In examples, this arrangement can provide an improvement over current technologies by, for example, helping reduce testing time and/or the consistency of results and analysis over multiple tests. Further, a user can perform two different testing protocols on the same biological testing sample at once. Further, a user can also perform tests with up to four different reservoir heights at once.

In some examples, the housing 402 comprises one or more fasteners configured to interface and/or couple the housing 402 to a sheet 406 to assemble the cartridge 400. For instance, in some examples, a sheet 406 can include one or more apertures compatible with the one or more fasteners. In some example configurations, such as the configuration shown in FIG. 4A, the housing 402 can include four fasteners, but many example configurations of fasteners are possible. In some examples, the fasteners include one or more datum pads and/or one or more heat stakes. In some alternative example embodiments, the housing 402 and sheet 406 can be coupled to each other via an adhesive (e.g., a UV cured adhesive).

In examples, the housing 402 includes optically transparent materials suitable for imaging. For instance, some example materials suitable for the housing 402 can include, but are not limited to, glass, acrylic, polystyrene, polypropylene, poly(methyl methacrylate) (PMMA), cyclic olefin copolymer (COC), and cyclic olefin polymer (COP). Many example materials are possible. In example embodiments, the optically transparent housing 402 helps reduce autofluorescence from the housing 402 to provide a clear fluorescent background for imaging.

In example embodiments, sheet 406 can include a label including a machine-readable identifier (e.g., a data matrix code, a barcode, a QR code, etc.). Further, in example embodiments, the label may be a laser-etched label, a printed label, and/or an adhesive label (e.g., a sticker), among other possibilities. In a further aspect, in examples, the QR code may be used to identify one or more medical records associated with sample, the patient, the imaging machine, and/or the testing facility, among other possibilities. For example, the QR code may be used as a verification protocol between the imaging device and the cartridge (e.g., an electronic handshake protocol) to ensure the cartridge is the proper cartridge for the imaging device. Other examples are possible. In examples, the sheet 406 includes optically transparent materials suitable for imaging. For instance, some example materials suitable for the sheet 406 can include, but are not limited to, glass, acrylic, polystyrene, polypropylene, PMMA, COC, and/or COP. Many example materials are possible. In some example embodiments, the material of the sheet 406 may be the same, or similar, to the material of the housing 402. In other examples, the material of the sheet 406 and the material of the housing 402 can be different from one another.

Turning to FIG. 4B, like FIG. 3, FIG. 4B illustrates an alternative view of the cartridge 400 illustrated in FIG. 4A and illustrates both reservoirs comprising two vents, including first vent 418A, second vent 418B, third vent 418C, and fourth vent 418D. In example embodiments, first vent 418A, second vent 418B, third vent 418C, and fourth vent 418D may promote fluid communication within each reservoir, including by promoting a fluid (e.g., a biological testing sample) dispersing throughout the reservoir after being inserted and dispersed into each reservoir's respective port. Other examples are possible.

Turning to FIG. 3C, FIG. 4C illustrates a cross-sectional view of the assembled cartridge 400, and includes housing 402, gasket 404, sheet 406, first port 408A, first reservoir 410A, first recess 412A, second recess 414A, and first vent 418A, with a fluid biological testing sample 420 disposed therein. As illustrated in FIG. 4C, the reservoirs comprise vents may help promote fluid dispersion and movement throughout the reservoir and avoid backflow of the biological testing sample once inserted into the cartridge 400 via first port 408. Additionally, as described above, FIG. 4C illustrates that the full volume of the fluid biological testing sample 420 has been transferred from the container to cartridge 400, which improves the consistency and accuracy of one or more testing protocols on the fluid biological testing sample 420, including by dispersing the fluid biological testing sample 420 evenly across the entire reservoir in cartridge 400.

Furthermore, as shown in FIG. 4C, because each first reservoir 410A contain two different recess heights, first recess 412A and second recess 414A, a user can perform testing protocols (e.g., imaging) at multiple depths within the same reservoir on the same the sample. For example, a user may perform a first analysis of a biological testing sample (e.g., capture a first set of images) in reservoir 410A at the shallow portion (corresponding to first recess 412A) and a second analysis of the same biological testing sample (e.g., capture a second set of images) in reservoir 410A at the deeper portion (corresponding to second recess 414A). In examples, this arrangement can provide an improvement over current technologies by, for example, helping reduce testing time and/or the consistency of results and analysis over multiple tests. Further, a user can perform two different testing protocols on the same biological testing sample at once.

Additionally, in example embodiments, the height of the first recess 412A can range from approximately 50 microns to approximately 500 microns inclusive of the endpoints, including, for example approximately 200 microns. Further, in example embodiments, the height of the second recess 414A can range from approximately 1.0 millimeter to approximately 3.0 millimeters inclusive of the endpoints, including, for example approximately 2.2 millimeters. For instance, in one example, the height of the first recess 412A can be 200 microns and the height of the second recess 414A can be 2.2 millimeters. Additionally, in an example embodiment, as illustrated in FIGS. 4A and 4C, the portion of reservoir between the first recess 412A and the second recess 414A may have a sloped surface so that a testing sample (e.g., fluid biological testing sample 420) may disperse and/or otherwise move more freely within the reservoir. Many example reservoir depths and configurations are possible.

Furthermore, different heights, volumes, and configurations of a reservoir can have advantages depending on the type of testing performed and/or the type of biological testing sample being tested. For instance, images captured of a biological testing sample having a high cell concentration can be more clear and easier to interpret in a reservoir with a lower height than images of the same biological testing sample in a reservoir having a greater height, for example, due to crowding of cells. In another example, a biological testing sample having a low cell concentration may not have enough cells in the reservoir with the lower height to result in an adequate image for analysis, but can result in adequate images for testing in the reservoir with the higher height, for example, after settling.

Further, for imaging fluid samples (e.g., blood), a shallow reservoir having a smaller reservoir height and volume can be more beneficial to help facilitate dispersing the biological sample into a thinner, more even layer. As such, imaging of a fluid sample may be more uniform which can improve consistency, accuracy, and repeatability of tests. Alternatively, in examples where the biological sample includes a solid sample (e.g., ear wax, fecal matter), it may be more beneficial to have a higher reservoir height, and thus a greater reservoir height and volume, to allow sufficient space for the sample to be imaged and/or mix with one or more liquids (e.g., a diluent and/or stain), among other possibilities.

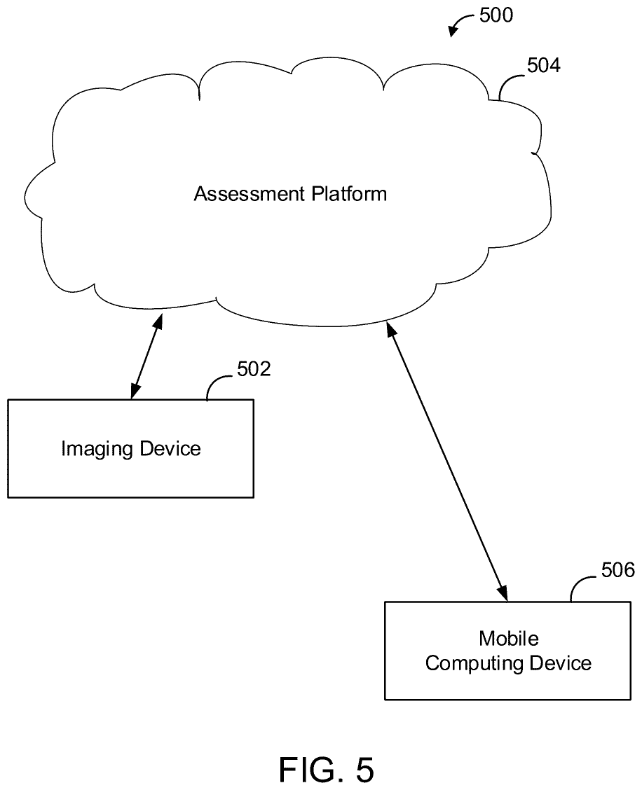

Now referring to FIG. 5, a computing system 500 configured for use with an imaging device 502 and a mobile computing device 506, according to an example embodiment. Example cartridges (e.g., cartridge 300 and/or cartridge 400) are compatible with an imaging device 502. An imaging device 502 includes a computing device, such as computing device 100. It should also be readily understood that computing device 100 and the imaging device 502, and all of the components thereof, can be physical systems made up of physical devices, cloud-based systems made up of cloud-based devices that store program logic and/or data of cloud-based applications and/or services (e.g., perform at least one function of a software application or an application platform for computing systems and devices detailed herein), or some combination of the two.

In any event, a computing system 500 can include various components, such as the computing device 100, imaging device 502, a cloud-based assessment platform.

The imaging device 502 and/or components thereof can perform various acts and/or functions (many of which are described above). Examples of these and related features will now be described in further detail.

The imaging device 502 may collect data from a number of sources. In one example, the imaging device 502 may collect data from a database of images related to assays and microscopic analyses of biological testing samples, including one or more images of biological samples. The images may be uploaded to an assessment platform 504 and characteristics of the images may be output to a mobile computing device 506.

In an example, assessment platform 504 may collect data from one or more sensors communicably coupled to the imaging device 502, such as an imaging sensor, concerning a particular biological testing sample. In such examples, the assessment platform 504 may identify a characteristic of the biological testing sample and transmit instructions to the mobile computing device 506 to cause a graphical user interface to display a graphical indication of the identified characteristic. In some examples, the assessment platform 504 may determine a characteristic of the biological testing sample by utilizing one or more of: (i) an artificial neural network, (ii) a support vector machine, (iii) a regression tree, or (iv) an ensemble of regression trees.

In another example, the imaging device 502 may collect data from one or more sensors communicably coupled to the imaging device, such as an imaging sensor, concerning a particular biological testing sample. In some examples, the assessment platform 504 may determine a characteristic of the biological testing sample by utilizing one or more of: (i) an artificial neural network, (ii) a support vector machine, (iii) a regression tree, or (iv) an ensemble of regression trees.

In some examples, images that are captured by the imaging device can be stored within a memory, such as a memory of computing device 100, to be subsequently analyzed.

In another example, the imaging device 502 may collect data from a plurality of sensors of the imaging device and superimpose the data. For example, assessment platform 504 may collect data in the form of monochromatic images (e.g., from different wavelength sources) from an imaging sensor of imaging device 502, and thermal data from a thermal imaging sensor of imaging device 502 and then overlay the thermal image with the monochromatic image. In some examples, assessment platform 504 may collect data from a sensor of the imaging device 502 and input data from a user of the mobile computing device 506 or a user of the imaging device 502. In one example, assessment platform 504 may transmit instructions to cause a graphical user interface to display a graphical indication of an identified characteristic along with the input data received from a user of the mobile computing device 506 or a user of the imaging device 502.

In one example, the imaging device 502 may train a machine learning model using data associated images of biological samples that share a characteristic with captured images of biological testing samples. The machine learning model may be trained using training data that shares a characteristic with a biological testing sample to be analyzed by the imaging device. Training the machine learning model may include inputting one or more training images into the machine learning model, predicting, by the machine learning model, an outcome of a determined condition of the one or more training images, comparing the at least one outcome to the characteristic of the one or more training images, and adjusting, based on the comparison, the machine learning model. For example, if a user is attempting to develop assays and microscopic analysis of blood samples to determine blood cell count, the machine learning model may be trained by inputting images of blood samples with known blood cell counts, predicting, by the machine learning model, a blood cell count of one or more training images, comparing the predicted blood cell count to the known blood cell count, and adjusting, based on the comparison, the machine learning model.

In some examples, the training data may include labeled input images (supervised learning), partially labeled input images (semi-supervised learning), or unlabeled input images (unsupervised learning). In some examples, training may include reinforcement learning.

The machine learning model may include an artificial neural network, a support vector machine, a regression tree, an ensemble of regression trees, or some other machine learning model architecture or combination of architectures.

The training data may include images of dry samples, images of fluid biological testing samples, images of solid biological samples, images of mixed fluid samples including biological samples and a stain configured to react in an aqueous solution, images of blank slides, synthetic, augmented images, or any combination thereof.

In some examples, the machine learning model of the imaging device 502 may be adjusted based on training such that if the outcome of a determined condition matches the characteristic of the training images, the machine learning model is reinforced and if the outcome of a determined condition does not match the characteristic of the training images, the machine learning model is modified. In some examples, modifying the machine learning model includes increasing or decreasing a weight of a factor within the neural network of the machine learning model. In other examples, modifying the machine learning model includes adding or subtracting rules during the training of the machine learning model.

Once the imaging device 502 has determined a characteristic of a biological sample in one or more images, the imaging device may transmit instructions that cause a computing device (e.g., the computing device 100) to display one or more graphical indications of the identified characteristic and/or the enhanced image. In example embodiments, the cartridge can be used for a variety of tests, including ear mite tests.

### EXAMPLES

The following examples illustrate the several volumes, mixtures, and concentrations of candidate diluents, surfactants, and reagents that can be prepared and tested to measure the efficacy of ear mite detection using the containers, cartridges, and imaging systems described herein.

For example, two conditions were identified that impact the transfer of ear mites in ear wax samples on a swab or other collection device to a liquid diluent in a container and then from the liquid diluent and ear mite mixture to a cartridge for microscopy evaluation. The first condition is the tendency of mites to float in a liquid (including liquid diluent), and the second condition is tendency of the ear mites to stick to solid surfaces (including those of the container and/or the cartridge).

### Example 1: Management of Transfer of Mites in a Sample Volume

For the first condition, management of floating mites, based on experimental outcomes of different volumes of the biological testing sample transferred from the container in which the biological testing sample was transferred to the cartridge, it was determined that the volume of the biological testing sample in container should match or approximately match the volume available in the reservoir of the cartridge. This consistency of volumes between the container and the reservoir of the cartridge promotes all of the components of the biological testing sample in container to be transferred from the container to the reservoir of the cartridge and therefore, reduces the deleterious effects of ear mites floating during transfer. To further promote this condition, the volume of diluent mixture can be configured such that even if a portion of the diluent mixture is transferred into the swab (e.g., absorbed by a cotton swab) during transfer of the biological sample into the diluent mixture, there is sufficient transfer diluent mixture and biological testing sample to the cartridge reservoir for evaluation. This volumetric match (or near match) between the container volume and the cartridge reservoir volume also reduces voids and/or significant bubbles in the viewing area of the reservoir that will be imaged during analysis (e.g., via a microscope and/or imaging device).

Further, in example embodiments, if the full matched volume from the container is transferred to the reservoir of the cartridge, then the reservoir will be filled with the biological testing sample and the reservoir can avoid overflow (e.g., because there is excess capacity in the cartridge other than the reservoir, including a port area of the cartridge).

Once present in a cartridge reservoir, an image of the sample can be obtained as described herein. Since sample mites tend to float, an image may be obtained at the top of the sample to capture the floating mites in the analysis of the sample.

### Example 2: Management of Mites Adhering to a Container Surface

For the second condition, even when all of the diluent is transferred to the cartridge, there are still surfaces in the container to which ear mites adhere, thereby keeping them in the container during and after transfer to the cartridge, therefore not in an area that can be imaged by the imaging system in connection with the cartridge. Two distinct experimental designs were evaluated to reduce the ear mites' adherence to the one or more surfaces of the container during transfer: (i) coating the interior surfaces of the container so that ear mites do not adhere to the interior surfaces of the container; and (ii) adding surfactants to the diluent mixture prior to transferring the biological sample into the container.

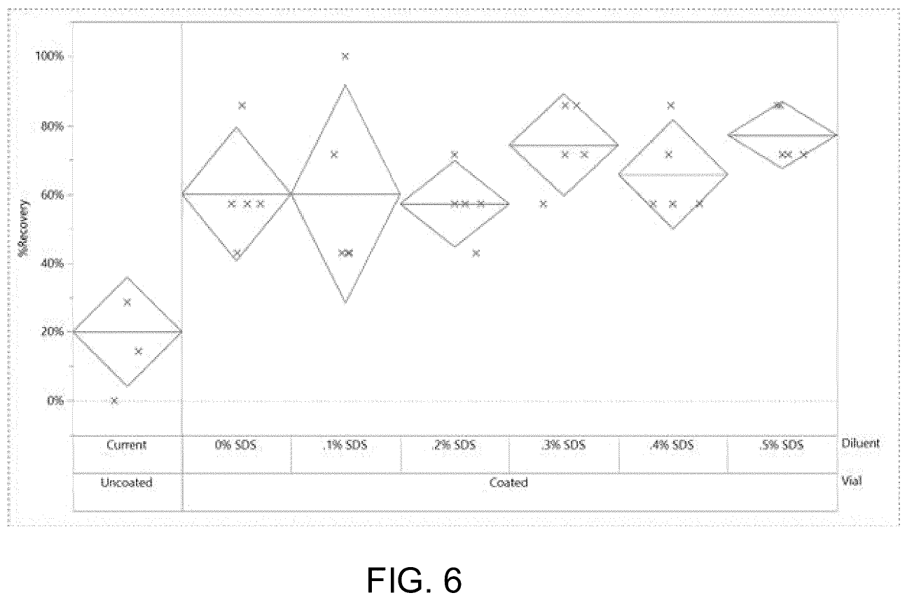

FIG. 6 illustrates the experimental results and recovery percentage of the ear mites from coated and uncoated containers and the use of various surfactants. In these experiments, the coating materials comprised silicon dioxide, but may also be silicon-based chemicals such as polysiloxanes. In these experiments, to prepare the coated container, the coating materials may be mixed with alcohol solution, and then one or more of the container components, including the container and/or the dispensing nozzle can be dipped into the solution and/or sprayed with the solution and left to dry for a period of time (e.g., at least 20 minutes at room temperature). As shown in FIG. 6, this coating method has proven successful in having a high transfer rate of ear mites from the container to the cartridge, but may add significant cost to the container. Thus, other experiments were undertaken to address ear mite transfer and recovery percentages.

As an alternative to coating a container, a diluent was used to extract elements from cotton swabs. The diluent included a surfactant that changes the surface tension of the sample to allow the mites in the sample to flow from the container into the reservoir of a test cartridge. The diluent included the surfactant (DOSS), two buffering agents to control pH, a salt to maintain osmolarity, a chelating reagent to avoid cell clumping, and a biocide agent to inhibit bacteria growth. Specific reagents are described below.

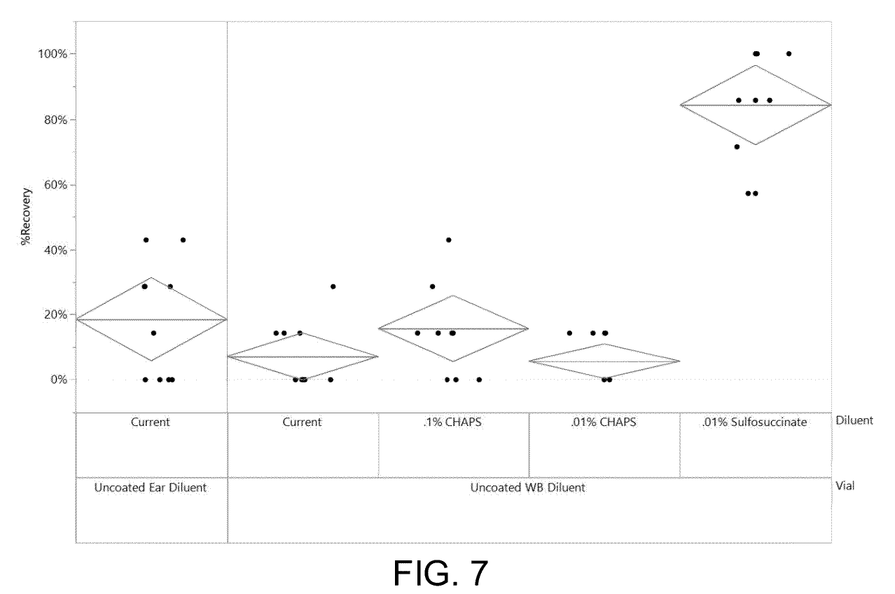

FIG. 7 illustrates the experimental results and recovery percentages of the ear mites based on various surfactants that were added to the diluent disposed in the container prior to transferring the biological testing sample (spiked with a known number of ear mites) to the cartridge. In these experiments, two surfactants were evaluated: DOSS and CHAPS (3-[(3-Cholamidopropyl)dimethylammonio]-1-propanesulfonate hydrate). As shown in FIG. 7, CHAPS did not result in an accurate determination of mites in uncoated sample containers. On the other hand, the use of DOSS resulted in a near complete detection of sample mites. In a similar experiment using TWEEN-20, results (not shown) were similar to CHAPS, which demonstrates that cationic and zwitterionic surfactants tested did not inhibit mite adhesion to surfaces.

In addition to showing the impact of coating a container, FIG. 7 shows that a second anionic surfactant (sodium dodecyl sulfate (SDS)), also demonstrated inhibition of adhesion to solid surfaces. While both anionic surfactants (DOSS and SDS) inhibited ear mite adhesion to surfaces, SDS concentration requirements were too high and negative impacts to cells were found (lysis of RBC and WBC). DOSS, on the other hand, was effective at inhibiting mite adhesion to surfaces and had little to no adverse effects on cells.

Moreover, combining DOSS as the surfactant in the diluent mixture with the matched concentration of disposable tube diluent and cartridge volume can provide a combined effect where over 75% of the ear mites released from the swab are then transferred to the cartridge.

### Example 3: Alternatives for the Determination of Living and Dead Ear Mites

Further, it was observed during these experiments that both living and dead ear mites will adhere to surfaces, which appears to indicate that the phenomenon is not an active function of the ear mite to grasp the solid surface, but instead is related to a physical force that allows the ear mite to adhere to the solid surface, which may be related to surface proteins or other chemicals naturally on the ear mite exoskeleton that supports adhesion to surfaces even if the ear mite is dead. This observed phenomenon could also be related to alternate forces, such as electrical attraction if the ear mite maintains a charge on its surface and can be attracted to or repelled from surfaces. Thus, there may be alternate potential methods of addressing this ear mite adhesion condition, but the present results separated each surfactant's efficacy on reducing adhesion based on their charge capacity (cationic having a positive charge, anionic having a negative charge, and zwitterionic having both charges).

Thus, based on these conditions and experiments, improved ear mite recovery was demonstrated with the proposed integration of a specific range of dioctyl sulfosuccinate in the diluent mixture and matching the volume between the reservoir of the cartridge and the volume of the biological testing sample (including the diluent, surfactant, reagent, and biological sample) in the container. As such, based on the experimentation detailed above, two example diluent mixtures are provided, depending on whether the ear mites are to be evaluated in the context of other cells, lysed or living, during the testing.

In the example embodiment pertaining to analyzing and/or otherwise imaging in living ear mites with other non-lysed cells (including RBC and WBC), the following chemical configuration for the biological testing sample was found to be efficacious:

| Water | 1 | L |
|---|---|---|
| DOSS | 0.01% | |
| Tris-HCl | 1.66 | g |
| Trizma*base | 2.2 | g |
| EDTA | 0.8 | g |
| Sodium Chloride | 8 | g |

In the example embodiment pertaining to analyzing and/or otherwise imaging in living ear mites with lysed cells (including lysed RBC and WBC), the following chemical configuration for the biological testing sample was found to be efficacious:

| Water | 1 | L |
|---|---|---|
| SDS | 0.05% | |
| Tris-HCl | 1.66 | g |
| Trizma*base | 2.2 | g |
| EDTA | 0.8 | g |
| Sodium Chloride | 8 | g |

When designing the experiments to address these conditions and support accurate ear cytology, steps can be taken to avoid destroying (e.g., lysing) any of the cells of interest in the biological testing sample. Generally, this design includes managing the osmolality and pH of the biological testing sample and being considerate of the type and concentration of other chemical components in the biological testing sample, such as surfactants. For some aspects of the disclosure, it is unnecessary to look sample components other than ear mites, which eliminates the need to avoid the destruction of other sample components. For example, SDS can lyse cellular components in the sample, while DOSS does not.

In addition, when incorporating stains into the reagents (e.g., bright field or fluorescent stains), reagents may be selected to optimized stain uptake. Example reagents include a lyophilized stain matrix including two buffering agents to control pH, a salt to maintain osmolarity, a fluorescent dye for staining intracellular nucleic acid, micron size polyacrylate-based beads for assisting imaging system focusing, an albumin for stabilized cells, and an excipient that supports the lyophilized matrix. An example reagent includes sodium chloride, potassium phosphate monobasic, sodium phosphate dibasic, Syto-13 dye, imaging polyacrylate-based focus beads, sucrose, and Bovine Serum Albumin.

### Example Methods and Aspects

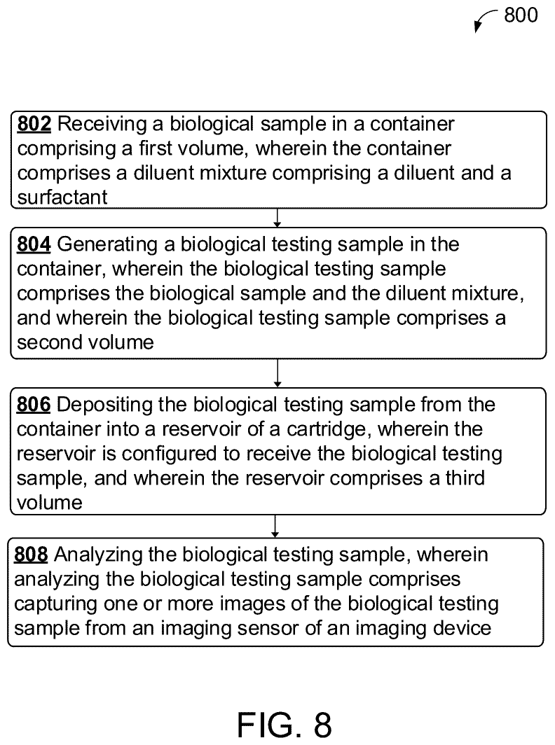

Now referring to FIG. 8, an example method of evaluating a biological sample comprising ear mites. Method 800 shown in FIG. 8 presents an example of a method of imaging a biological sample that could be used such as the example containers, cartridges, and/or imaging devices shown in FIGS. 2-5, for example. Further, devices or systems may be used or configured to perform logical functions presented in FIG. 8. In other examples, components of the devices and/or systems may be arranged to be adapted to, capable of, or suited for performing the functions, such as when operated in a specific manner. Method 800 may include one or more operations, functions, or actions as illustrated by one or more of blocks 802-808. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 802, method 800 involves receiving a biological sample in a container comprising a first volume, wherein the container comprises a diluent mixture comprising a diluent and a surfactant. In some examples, the container further comprises one or more extraction ribs configured to extract the biological sample. In some examples, the biological sample further comprises ear wax. In some examples, the biological sample further comprises one or more of the following: (i) blood; (ii) urine; (iii) saliva; (iv) fecal matter; (v) secretion; (vi) excretion; (vii) FNA; (viii) lavage fluids; (ix) body cavity fluids; (x) semen; (xi) ear wax; (xii) skin cells; (xiii) biopsied samples, (xiv) exotics; (xv) cultured cells; (xvi) bacteria; (xvii) worms; and (xviii) parasites. In some examples, the surfactant comprises an anionic surfactant. In some examples, the anionic surfactant comprises dioctyl sulfosuccinate. In some examples, the diluent mixture comprises about 0.005% to about 0.02% dioctyl sulfosuccinate, for example about 0.005%, 0.01%, 0.015% and 0.02% dioctyl sulfosuccinate. In another embodiment, the diluent mixture includes 0.05% to about 1.0% SDS, for example about 0.05%, 0.1%, 0.2%, 0.3%, 0.4%, 0.5%, 0.75% or 1.0% SDS. In some examples, the remainder of the diluent may be water or other biologically compatible liquid.

At block 804, method 800 involves generating a biological testing sample in the container, wherein the biological testing sample comprises the biological sample and the diluent mixture, and wherein the biological testing sample comprises a second volume. In some examples, the reagent comprises a liquid reagent. In some examples, the reagent comprises a solid reagent. In some examples, the solid reagent comprises a lyophilized reagent. In some examples, the first volume and the second volume are approximately the same volume. In some examples, each of the first volume and the second volume is approximately 0.5 milliliters.

At block 806, method 800 involves depositing the biological testing sample from the container into a reservoir of a cartridge, wherein the reservoir is configured to receive the biological testing sample, and wherein the reservoir comprises a third volume. In some examples, the first volume, the second volume, and the third volume are approximately the same volume. In some examples, each of the first volume, the second volume, and the third volume is approximately about 0.25 to about 0.75 milliliters, for example, 0.5 milliliters. In some examples, the container comprises a compliant material such that when compressed, the biological testing sample is dispensed from a dispensing nozzle of the container. In some examples, generating the biological testing sample in the container comprises agitating the biological sample, the diluent mixture, and the reagent in the container.

At block 808, method 800 involves analyzing the biological testing sample, wherein analyzing the biological testing sample comprises capturing one or more images of the biological testing sample from an imaging sensor of an imaging device. In some examples, analyzing the biological testing sample further comprises inputting the one or more images into one or more machine learning models, identifying, via the one or more machine learning models, one or more characteristics of the biological testing sample in the one or more images, and transmitting instructions that cause a graphical user interface to display the one or more characteristics of the biological testing sample in the one or more images.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A method of evaluating a biological sample comprising ear mites, the method comprising:
receiving the biological sample in a container comprising a first volume, wherein the container comprises a diluent mixture comprising a diluent and a surfactant;
generating a biological testing sample in the container, wherein the biological testing sample comprises the biological sample and the diluent mixture, and wherein the biological testing sample comprises a second volume;
depositing the biological testing sample from the container into a reservoir of a cartridge, wherein the reservoir is configured to receive the biological testing sample, and wherein the reservoir comprises a third volume; and
analyzing the biological testing sample, wherein analyzing the biological testing sample comprises capturing one or more images of the biological testing sample from an imaging sensor of an imaging device.

2. The method of claim 1, wherein the container further comprises one or more extraction ribs configured to extract the biological sample.

3. The method of claim 2, wherein the biological sample further comprises ear wax.

4. The method of claim 1, wherein the biological sample further comprises one or more of the following: (i) blood; (ii) urine; (iii) saliva; (iv) fecal matter; (v) secretion; (vi) excretion; (vii) FNA; (viii) lavage fluids; (ix) body cavity fluids; (x) semen; (xi) ear wax; (xii) skin cells; (xiii) biopsied samples, (xiv) exotics; (xv) cultured cells; (xvi) bacteria; (xvii) worms; and (xviii) parasites.

5. The method of claim 1, wherein the surfactant comprises an anionic surfactant.

6. The method of claim 5, wherein the anionic surfactant comprises dioctyl sulfosuccinate (DOSS) or sodium dodecyl sulfate (SDS).

7. The method of claim 6, wherein the diluent mixture comprises approximately 0.005% to 0.1% DOSS or approximately 0.05% to 1.0% SDS.

8. The method of claim 1, wherein the biological testing sample further comprises a liquid reagent.

9. The method of claim 1, wherein the biological testing sample further comprises a solid reagent.

10. The method of claim 9, wherein the solid reagent comprises a lyophilized reagent.

11. The method of claim 1, wherein the first volume and the second volume are approximately the same volume.

12. The method of claim 11, wherein each of the first volume and the second volume is approximately 0.5 milliliters.

13. The method of claim 1, wherein the first volume, the second volume, and the third volume are approximately the same volume.

14. The method of claim 13, wherein each of the first volume, the second volume, and the third volume is approximately 0.5 milliliters.

15. The method of claim 1, wherein the container comprises a compliant material such that when compressed, the biological testing sample is dispensed from a dispensing nozzle of the container.

16. The method of claim 1, wherein generating the biological testing sample in the container comprises agitating the biological sample and the diluent mixture in the container.

17. The method of claim 1, wherein analyzing the biological testing sample further comprises:
inputting the one or more images into one or more machine learning models;
identifying, via the one or more machine learning models, one or more characteristics of the biological testing sample in the one or more images; and
transmitting instructions that cause a graphical user interface to display the one or more characteristics of the biological testing sample in the one or more images.

18. A non-transitory computer-readable medium, having stored thereon program instructions that, upon execution by a computing device, cause the computing device to perform a set of operations for evaluating a biological sample comprising ear mites, the set of operations comprising:
receiving the biological sample in a container comprising a first volume, wherein the container comprises a diluent mixture comprising a diluent and a surfactant;
generating a biological testing sample in the container, wherein the biological testing sample comprises the biological sample and the diluent mixture, and wherein the biological testing sample comprises a second volume;
depositing the biological testing sample from the container into a reservoir of a cartridge, wherein the reservoir is configured to receive the biological testing sample, and wherein the reservoir comprises a third volume; and
analyzing the biological testing sample, wherein analyzing the biological testing sample comprises capturing one or more images of the biological testing sample from an imaging sensor of an imaging device.

19. The non-transitory computer-readable medium of claim 18, wherein the container further comprises one or more extraction ribs configured to extract the biological sample, and wherein the biological sample comprises ear wax.

20. The non-transitory computer-readable medium of claim 18, wherein the surfactant comprises dioctyl sulfosuccinate (DOSS) or sodium dodecyl sulfate (SDS).
